(19) United States
(12) Patent Application Publication
(10) Pub. No.: US 2014/0212355 A1
(43) Pub. Date: Jul. 31, 2014

(54) TRANS-ARTERIAL DRUG DELIVERY

(71) Applicant: ABBOTT CARDIOVASCULAR SYSTEMS INC., Santa Clara, CA (US)

(72) Inventors: Mikael TROLLSAS, San Jose, CA (US); Syed Hossainy, Hayward, CA (US); John Stankus, Campbell, CA (US); Paul Consigny, San Jose, CA (US)

(73) Assignee: Abbott Cardiovascular Systems Inc., Santa Clara, CA (US)

(21) Appl. No.: 13/752,281

(22) Filed: Jan. 28, 2013

(51) Int. Cl. A61K 9/00

(52) U.S. Cl. CPC A61K 9/0019
USPC 424/1.29; 424/484; 424/145.1; 424/486; 424/152.1; 514/350; 514/407; 514/449; 514/291; 514/301

(57) ABSTRACT

It is provided herein methods, devices, and compositions for trans-arterial local delivery of therapeutic agent for the treatment of liver cancers.

### FIELD OF THE INVENTION

This invention relates to methods, devices, and compositions for local delivery of therapeutic agents for the treatment of liver cancers.

### BACKGROUND OF THE INVENTION

The liver is a vital organ present in vertebrates and some other animals. It plays a major role in metabolism and has a number of functions in the body, including glycogen storage, decomposition of red blood cells, plasma protein synthesis, hormone production, and detoxification. The liver is connected to two large blood vessels, the hepatic artery and the portal vein. The hepatic artery carries blood from the aorta, whereas the portal vein carries blood containing digested nutrients from the entire gastrointestinal tract and also from the spleen and pancreas. These blood vessels subdivide into capillaries, which then lead to a lobule. Each lobule is made up of millions of hepatic cells which are the basic metabolic cells. The liver gets a dual blood supply from the hepatic portal vein and hepatic artery.

The liver can be affected by primary liver cancer which arises in the liver itself, or by cancer which forms in other sites and then spreads to the liver. Most cancers in the liver are secondary or metastatic, meaning they start elsewhere in the body and spread to the liver. Hepatocellular carcinoma (HCC) is one of the most common liver cancers. Depending on the stage of the cancer, the arterial blood supply to the cancer can be blocked by the use of embolic beads delivered trans-arterially with a catheter. The embolic beads can deliver anti-cancer drug locally, which can be radioactive.

Recently it has been discovered that the embolic beads create hypoxia in the treated tumor tissue, which leads to up-regulation of vascular endothelial growth factor (VEGF) and stimulation of angiogenesis as a result. Recent studies have, therefore, involved systemically delivered drugs such as Sorafenib and Avastin to prevent angiogenesis in the tumor. However, systemic delivery of drug has drawbacks such as systemic toxicity and reduced bioavailability at the disease site. Additionally, some drugs are difficult to formulate for systemic delivery. Therefore there is a need to deliver drugs to prevent angiogenesis without drawbacks of systemic delivery.

### INCORPORATION BY REFERENCE

All publications and patent applications mentioned in this specification are hereby incorporated by reference to the same extent as if each individual publication or patent application was specifically and individually indicated to be incorporated by reference, and as if each said individual publication or patent application was fully set forth, including any figures, herein.

### SUMMARY OF THE INVENTION

In one aspect, provided herein is a method of treating a liver cancer in a subject in need thereof. The method comprises the step of deploying a bioresorbable polymer scaffold in the lumen of a blood vessel that directly services a diseased liver or the cancer tissue therein, wherein the bioresorbable polymer scaffold comprises a polymer substrate and optionally a coating upon the substrate, wherein a first therapeutic agent is embedded or impregnated in the substrate, the coating if present, or both; wherein a therapeutically effective amount of the first therapeutic agent is released from the scaffold upon the deployment thereof over a period of time.

In one aspect, provided herein is a drug eluting device. The device comprises (1) a bioresorbable polymer scaffold which comprises a polymer substrate and optionally a coating upon the substrate, (2) a first therapeutic agent selected from the group consisting of an anti-VEGF antibody, an anti-EGFR antibody, a small molecule anti-angiogenesis drug, and any combination thereof; and (3) optionally a second therapeutic agent selected from the group consisting of an anti-proliferative agent, an anti-inflammatory agent, an anti-neoplastic agent, and any combination thereof. The therapeutic agents are embedded or impregnated in the polymer substrate, the coating if present, or both.

In one aspect, provided herein is a pharmaceutical composition for trans-arterial delivery of a therapeutic agent to a blood vessel. The composition comprises (1) a first therapeutic agent which is an anti-angiogenesis agent selected from the group consisting of an anti-VEGF antibody, an anti-EGFR antibody, a small molecule anti-angiogenesis drug, and any combination thereof, (2) optionally a second therapeutic agent selected from the group consisting of anti-proliferative agents, anti-inflammatory agents, anti-neoplastic agent, and any combination thereof, and (3) a polymeric carrier thereof. The polymeric carrier could be an injectable hydrogel comprising one or more different polymer molecular structures that could be inert or having structures that would allow them to react with each other if activated. To allow the polymers to react with each other the composition would also comprise (4) an activation buffer or agent. An additional aspect of the invention would be to have the therapeutic agent or agents delivered inside a polymeric particle and optionally have the polymeric particle delivered inside an injectable hydrogel comprising one or more different polymer structures that could be inert or have a structure that allows them to react with each other if activated. To allow the polymers to react with each other the composition would also comprise (4) an activation buffer or agent. It is also provided herein a method of treating liver cancer. The method comprises delivering the above mentioned pharmaceutical composition trans-arterially to a blood vessel that directly services the diseased liver or the cancer tissue therein.

In one aspect, it is provided a method for the treatment of a liver cancer, comprising providing a composition that comprises a crosslinkable component, providing a therapeutic agent in a pharmaceutically effective amount to the composition, rendering the crosslinkable component crosslink to form a hydrogel, and delivering the hydrogel containing the therapeutic agent to a blood vessel that directly services the diseased liver or the cancer tissue therein.

In some embodiments, the local or trans-arterial delivery of therapeutic agent is combined with a systemic delivery of therapeutic agent, wherein the two modes of delivery are additive or synergistic to each other. Exemplary systemic delivery includes oral administration and intravenous injection or infusion.

### BRIEF DESCRIPTION OF THE DRAWINGS

FIG. 1 depicts an exemplary stent scaffolding.

FIG. 2 depicts an exemplary stent pattern shown in a planar or flattened view.

FIG. 3 depicts an exemplary injectable hydrogel delivery system.

FIGS. 4 (4A and 4B) depicts chemical structures of exemplary multi-functional PEGs that can be used to form injectable hydrogel system.

FIGS. 5 (5A and 5B) depicts formation of 4+4 intermediates of crosslinked multi-functionalized PEGs.

### DETAILED DESCRIPTION OF THE INVENTION

### Definition

It is understood that use of the singular throughout this application including the claims includes the plural and vice versa unless expressly stated otherwise. That is, “a” and “the” are to be construed as referring to one or more of whatever the word modifies. Non-limiting examples are: “a therapeutic agent” which is understood to include one such agent, two such agents or, under the right circumstances, as determined by those skilled in the treatment of diseased tissues, even more such agents unless it is expressly stated or is unambiguously obvious from the context that such is not intended. Likewise, “a biodegradable polymer” refers to a single polymer or a mixture of two or more polymers unless, again, it is expressly stated or absolutely obvious from the context that such is not intended.

As used herein, “substantial” or “substantially” means that the object of the adjective or adverb is not a perfect example of such object but would be immediately envisaged by the skilled artisan to warrant the general designation. That is, when modified by the word “substantially,” it is understood that the object of the modifier would be considered close enough to be recognized by those of ordinary skill in the art as being within the general genus of such objects.

The use of other words or approximation herein, such as “about” or “approximately” when used to describe numerical values or ranges likewise are understood to mean that those skilled in the art would readily consider a value different from the exact number or outside the actual range to be close enough to be within the aegis of that number or range. At the very least, “about” or approximately is understood to mean±15% of a given numerical value or range starting and ending point.

As used herein, “treating” or “treatment” refers to the administration of a therapeutically effective amount of a therapeutic agent to a patient afflicted with a diseased tissue.

A “subject” refers to any species that might benefit from treatment using the method herein but at present preferably a mammal and most preferably a human being.

As used herein, a “tissue,” refers to any group of cells that in the aggregate perform the same function.

As used herein, a “liver cancer” refers to a primary liver cancer or a secondary or metastatic liver cancer. The term “liver cancer” may be used interchangeably with the term “liver tumor.”

As used herein, a “diseased liver” refers to a liver that is affected by a cancer.

As used herein, a “therapeutic agent” refers to any substance that, when administered in a therapeutically effective amount to a patient, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. The term “therapeutic agent” may refer to a biologic or a small molecule drug and therefore may be used with the term “biologic” or “drug” interchangeably in some instances.

As used herein, “biologic” refers to a medicinal preparation created by a biological process. For example, an antibody may be referred as a biologic.

A “therapeutically effective amount” refers to that amount of a therapeutic agent that will have a beneficial effect, which may be curative or palliative, on the health and well-being of the patient so afflicted. A therapeutically effective amount may be administered as a single bolus, as intermittent bolus charges, as short, medium or long term sustained release formulations or as any combination of these. As used herein, short-term sustained release refers to the administration of a therapeutically effective amount of a therapeutic agent over a period of about an hour to about 3 days. Medium-term sustained release refers to administration of a therapeutically effective amount of a therapeutic agent over a period of about 3 days to about 4 weeks and long-term refers to the delivery of a therapeutically effective amount over any period in excess of about 4 weeks, but in particular at present about 4 weeks to about a year. A therapeutically effective amount can also be released from an implantable drug eluting device such as a drug eluting stent.

As provided herein, a “bioresorbable polymer scaffold” refers to a structure made of one or more bioresorbable polymers. In some embodiments, the bioresorbable polymer scaffold is an implantable device, such as a stent. In some embodiments, the bioresorbable polymer scaffold comprises a polymer substrate and a coating deposited upon the substrate.

Hydrogels are three-dimensional, crosslinked networks of water soluble polymers. Hydrogels can be made from virtually any water soluble polymers encompassing a wide variety of chemical compositions. Crosslinking strategies that can be used include UV photo-polymerization and various chemical and physical crosslinking techniques. Chemical crosslinking techniques include use of pre-functionalized polymer with reactive functional groups and/or crosslinkers. Physical crosslinking techniques include triggering change in pH, temperature, light, ionic strength, etc. in the environment of the polymer. Physical crosslinking techniques also include the use of morphological changes, such as crystallinity, precipitation, or the use of hydrogen bonding.

By “bioabsorbable” or “bioresorbable,” it is meant that the polymer, the polymeric scaffold, or the polymeric matrix can be absorbed by bioabsorption.

As used herein, the term “biodegradation” includes all means by which a polymer can be disposed of in a patient's body, which includes bioabsorption, bioresorption, etc. Degradation occurs through hydrolysis, enzymatic reactions, and other chemical reactions. Biodegradation can take place over an extended period of time, for example over 2-3 years. The term “biostable” means that the polymer does not biodegrade or bioabsorb under physiological conditions, or the polymer biodegrades or bioabsorbs very slowly over a very long period of time, for example, over 5 years or over 10 years.

As used herein, a “lumen” refers to a cavity of a tubular organ such as a blood vessel. In the embodiments of the present invention, a lumen refers to a cavity of a blood vessel such as an artery.

As used herein a “carrier” refers to the substance that constitutes the continuous phase of a drug eluting device or a pharmaceutical composition. In a drug eluting device, a carrier can mean the bioabsorbable scaffold.

As used herein, “biocompatible” refers to a property of a material characterized by it, or its physiological degradation products, being not, or at least minimally, toxic to living tissue; not, or at least minimally and reparably, otherwise injurious living tissue; and/or not, or at least minimally and controllably, causative of an immunological reaction in living tissue.

As used herein, “catheter” refers to a tube that can be inserted into a body cavity, duct, or vessel. Catheters allow administration of fluids or gases or access by surgical instruments. In most uses, a catheter is a thin, flexible tube (“soft” catheter).

By “trans-arterial delivery” or “deliver trans-arterially” it is meant that a scaffold or hydrogel comprising a therapeutic agent is delivered through the arteries to any cancer tissue, or to a diseased liver or the cancer tissue therein, e.g., through an artery that directly services the diseased liver or the cancer tissue therein.

By “directly services” it is meant that blood flowing through the artery proceeds in one direction only through the labyrinthine maze comprising artery→arterioles→metarterioles→capillaries→postcapillary venules→venules→vein. As used herein, an artery that directly services the diseased tissue refers to an artery sufficiently near the diseased tissue that blood entering that artery must proceed by means of the circulatory system into and through the diseased tissue such that the bioresorbable polymer scaffold of this invention are entrapped entirely or at least predominantly in the target diseased tissue. Such arteries include, without limitation, the hepatic artery.

Physiological conditions merely refer to the physical, chemical and biochemical milieu that constitutes the mammalian body and includes, without limitation, pH, temperature, enzymes and the presence of destructive cells such as phagocytes.

### Liver Cancer and Treatment Thereof

This invention relates to methods, devices, and compositions for trans-arterial local delivery of therapeutic agent for the treatment of liver cancers.

Local drug delivery can maintain a therapeutically effective local exposure and reduced systemic exposure (e.g. Cmax and AUC) to minimize potential side effects (e.g. GI perforation, incomplete wound healing, bleeding problems) to the patient.

In the present invention, an implantable drug eluting device such as an implantable drug eluting stent or a pharmaceutical composition such as drug-containing microparticles or nanoparticles, drug-containing beads, a drug-containing hydrogel, or any combination thereof is used to deliver one or more therapeutic agents to a liver cancer tissue locally and trans-arterially. The methods of local delivery are adapted to the use of the implantable drug eluting device and the pharmaceutical composition. Here, liver cancer includes primary and secondary or metastatic liver cancers.

In some embodiments, the present method, device, and composition are used to treat a liver cancer by trans-arterial local delivery of one or more therapeutic agents. In each of the above described conditions, an anti-angiogenesis agent, an anti-cancer agent of other type, or any combination thereof are delivered directly into the diseased liver or the cancer tissue therein. Local delivery of the therapeutic agent into the liver has the advantage of exposing the diseased liver and thus the cancer to a high concentration of the therapeutic agent, thus minimizing systemic toxicity and side effects.

In one aspect of the invention, it is provided a method of treating a liver cancer in a subject in need thereof. The method comprises deploying a bioresorbable polymer scaffold in the lumen of a blood vessel that directly services the diseased liver or the cancer tissue therein. The bioresorbable polymer scaffold is embedded or impregnated with one or more therapeutic agents. A therapeutically effective amount of the therapeutic agent is released from the scaffold upon the deployment thereof over a period of time. The therapeutic agent is released directly to the cancer tissue or into the blood supply to the cancer tissue.

The bioabsorbable polymer scaffold comprises a polymer substrate and optionally a coating upon the substrate, wherein the substrate, or the coating if present, or both comprise a first therapeutic agent.

In some embodiments, the bioabsorbable polymer scaffold is a stent.

In some embodiments, the blood vessel is a hepatic artery. In some embodiments, the blood vessel is a branched artery of the hepatic artery that is connected with the diseased liver. In some embodiments, the blood vessel is a hepatic artery proximal to a diseased liver or a cancer tissue therein.

The bioresorbable polymer scaffold may be deployed by varying means. In some embodiments, a bioresorbable polymer scaffold is inserted directly into a hepatic artery. In some embodiments, a bioresorbable polymer scaffold is inserted into a peripheral artery and threaded through until it is intersects the hepatic artery. In some embodiments, a bioresorbable polymer scaffold is inserted in a surgically created cavity in the liver. In some embodiments, the scaffold is deployed by inserting the scaffold through small lumens using a catheter and transporting it to the treatment site. Deployment includes expanding the scaffold to a larger diameter once it is at the desired location.

Once expanded, the scoffold must maintain its expanded diameter during a time required for treatment in spite of the various forces that may come to bear on it. In addition, the scaffold must possess sufficient flexibility with a certain resistance to fracture.

In some embodiments, the bioresorbable polymer scaffold is a hydrogel. In some embodiments, the blood vessel is a hepatic artery. In some embodiments, the blood vessel is a branched artery of the hepatic artery that is connected with the diseased liver. In some embodiments, the blood vessel is a hepatic artery proximal to a diseased liver or a cancer tissue therein. The hydrogel may be delivered by varying means. In some embodiments, a hydrogel is inserted directly into a hepatic artery. In some embodiments, a hydrogel is inserted into a peripheral artery and threaded until it intersects the hepatic artery.

In some embodiments, the liver cancer is hepatocellular carcinoma (HCC). In some embodiments, the liver cancer is colorectal liver metastasis. In some embodiments, the liver cancer is or heptoblastoma.

In some embodiments, the method further comprises a step of delivering embolic beads to the tumor, wherein the embolic beads carry a radioisotope, a radioactive anti-cancer drug, a chemotherapy drug, or a biologic. The embolic beads can be bioabsorbable as well. The embolic beads can be delivered either prior to or after the deployment of the bioresorbable polymer scaffold. Preferably the embolic beads are delivered prior to deploying the bioresorbable polymer scaffold. In some embodiments, the embolic bead may be delivered together with a hydrogel or an in situ forming hydrogel.

In some embodiments, the cancer tissue is in a state of hypoxia. The state of hypoxia may be due to blockage of arterial blood supply by embolic beads. The embolic beads have been administered previously in a separate treatment, or as a part of the present treatment.

In some embodiments, the first therapeutic agent is an anti-angiogenesis agent. The anti-angiogenesis agent includes an anti-VEGF (vascular endothelial growth factor) antibody, an anti-EGFR (Epidemal Growth Factor Receptor) antibody, a small molecule anti-angiogenesis drug, and any combination thereof. In some embodiments, the anti-VEGF antibody is bevacizumab (e.g., Avastin by Genentech/Roche). In some embodiments, the anti-EGRF antibody is ABT-806. In some embodiments, the small molecule drug is sorafenib (brand name Nexavar), or linifanib (also known as ABT-869), or ABT-348. As used herein, “ABT” indicates the therapeutic agents developed or made available by Abbott Laboratories.

Linifanib (ABT-869) is a receptor tyrosine kinase (RTK) inhibitor and is a potent inhibitor of members of the vascular endothelial growth factor (VEGF) and platelet-derived growth factor (PDGF) receptor families. Linifanib (ABT-869) has the following chemical structure:

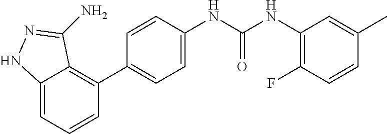 [SMILES: Cc1ccc(F)c(NC(=O)Nc2ccc(-c3cccc4[nH]nc(N)c34)cc2)c1]

Sorafenib is a small molecule inhibitor of several tyrosine protein kinases (VEGFR and PDGFR) and Raf kinases. It has the following chemical structure:

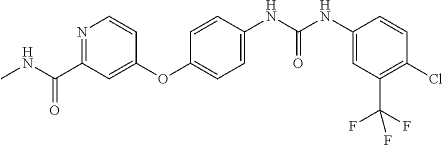 [SMILES: CNC(=O)c1cc(Oc2ccc(NC(=O)Nc3ccc(Cl)c(C(F)(F)F)c3)cc2)ccn1]

In some embodiments, the anti-angiogenesis agent is ABT-348 or ABT-993.

ABT-348 is an ATP-competitive inhibitor of Aurora kinase and has a potent binding activity against the VEGFR/PDGFR families and the SRC family of cytoplasmic tyrosine kinases, which leads to potent inhibition of VEGF-stimulated endothelial cell proliferation.

In some embodiments, the substrate or the coating or both further comprise a second therapeutic agent. In some embodiments, the second therapeutic agent is an mTOR inhibitor. In some embodiments, the second therapeutic agent is an anti-proliferative agent, an anti-inflammatory agent, or an anti-neoplastic agent. Specific second therapeutic agents include but not limited to zotarolimus, everolimus, sirolimus, tacrolimus, biolimus, deforolimus, SAR-943, halofuganone, an anti-TNF agent, a BCL-2 inhibitor and combination thereof.

SAR-943 (32-deoxo rapamycin) is a proliferation signal inhibitor via interaction with the mammalian target of rapamycin (mTOR). SAR-943 (Novartis) is of particular note as it is 10 to 100 fold more potent than zotarolimus. Given the greater potency of SAR-943, one could use less drug to obtain the same amount of inhibition or use the same or more drug to extend the duration of release.

Specific anti-TNF agents include monoclonal antibody such as infliximab (Remicade), adalimumab (Humira), certolizumab pegol (Cimzia), and golimumab (Simponi), and etanercept (Enbrel). Specific BCL-2 inhibitors include antisense oligonucleotide drug Genasense (G3139), ABT-737 and ABT-199.

In some embodiments, the substrate of the scaffold comprises a bioabsorbable polymer selected from the group consisting of poly(DL-lactide), poly(L-lactide), poly(D-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof, and a combination thereof.

In some embodiments, the substrate comprises a bioabsorbable polymer that is a poly(L-lactide), poly(L-lactide-co-glycolide), or poly(L-lactide-co-D,L-lactide).

In some embodiments, the coating is a polymeric matrix comprising a bioabsorbable polymer selected from the group consisting of poly(DL-lactide), poly(L-lactide), poly(D-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof, and a combination thereof.

In some embodiments, the polymer matrix comprises a bioabsorbable polymer that is poly(D,L-lactide), poly(lactide-co-caprolactone), or poly(glycolide-co-caprolactone).

The substrate or the polymer matrix is partially or completely made of the bioabsorbable polymer mentioned above. The substrate or the polymer matrix may contain about 50% to 100%, for example, about 50%, about 60%, about 70%, about 80%, about 90%, or about 100% of the polymer mentioned above. The rest is made up by another biocompatible polymer suitable for fabricating a substrate or a polymer matrix in combination with the polymer mentioned above, or other components such as therapeutic agents, inorganic fillers, or combination thereof.

The loading of the therapeutic agents may vary. In the substrate, the ratio of polymer and therapeutic agent by weight may vary between 500:1 and 50:1, for example 400:1, 300:1, 200:1, 100:1, 90:1, 80:1, 70:1 and 60:1. In the coating, the ratio of polymer and therapeutic agent by weight may vary between 10:1 and 1:10, for example, 9:1, 7:1, 5:1, 3:1, 1:1, 1:3, 1:5, 1:7, and 1:9.

The first therapeutic agent can have a controlled release profile. The second therapeutic agent can also have a controlled release profile.

In one aspect of the invention, it is provided a drug eluting device which comprises a bioresorbable polymer scaffold, a first therapeutic agent, and optionally a second therapeutic agent. The bioresorbable polymer scaffold comprises a polymer substrate and optionally a coating deposited upon the substrate. The therapeutic agents are embedded or impregnated in the polymer substrate, the coating if present, or both.

The coating can be a polymeric matrix deposited upon the polymer substrate. The coating can have one or more layers in any combination, including but not limited to a primer layer, which may improve adhesion of subsequent layers on the implantable substrate or on a previously formed layer; (b) a reservoir layer, which may comprise a polymer and a therapeutic agent or, alternatively, a polymer free agent; (c) a topcoat layer, which may serve as a way of controlling the rate of release of an agent; and (d) a biocompatible finishing layer, which may improve the biocompatibility of the coating. The polymer matrix and polymer substrate can be completely absorbed by the body, preferably at different rate.

The first therapeutic agent is an anti-VEGF antibody, an anti-EGFR antibody, or a small molecule anti-angiogenesis drug. In some embodiments, the anti-VEGF antibody is bevacizumab. In some embodiments, the anti-EGRF antibody is ABT-806. In some embodiments, the small molecule drug is sorafenib or linifanib (ABT869) or ABT-348.

In some embodiments, the drug eluting device comprises a second therapeutic agent selected from an anti-proliferative agent, an anti-inflammatory agent, and an anti-neoplastic agent. Specific second therapeutic agents include paclitaxel, zotarolimus, everolimus, sirolimus, tacrolimus, biolimus, deforolimus, SAR-943, halofuganone, an anti-TNF agent, and combination thereof.

In some embodiments, the drug eluting device is a stent.

The first therapeutic agent can have a controlled release profile. The second therapeutic agent can also have a controlled release profile.

### Therapeutic Agent Delivery Composition and Method

In one aspect of the invention, it is provided a pharmaceutical composition for trans-arterial local delivery of one or more therapeutic agents. The composition comprises a first therapeutic agent, optionally a second therapeutic agent, and a polymeric carrier thereof.

The first therapeutic agent is an anti-angiogenesis agent including an anti-VEGF antibody, an anti-EGFR antibody, a small molecule anti-angiogenesis drug, and any combination thereof. In some embodiments, the anti-VEGF antibody is bevacizumab. In some embodiments, the anti-EGRF anti-body is ABT-806. In some embodiments, the small molecule drug is sorafenib or linifanib (ABT869) or ABT-348.

In some embodiments, the pharmaceutical composition comprises a second therapeutic agent including anti-proliferative agents, anti-inflammatory agents, and anti-neoplastic agent. Specific second therapeutic agent includes paclitaxel, zotarolimus, everolimus, tacrolimus, biolimus, sirolimus, deforolimus, SAR-943, halofuganone, or an anti-TNF agent.

In some embodiments, the carrier is polymeric microparticles or nanoparticles. Microparticles refer to particles between about 0.1 μm and about 100 μm in diameter. Nanoparticles refer to particles between about 100 nm and about 10,000 nm in diameter. Fine nanoparticles refer to particles between about 100 nm and about 2500 nm in diameter.

The first therapeutic agent is embedded or impregnated in the polymeric microparticles or nanoparticles. In some embodiments, the polymeric microparticles or nanoparticles comprise a bioabsorble polymer. The bioabsorbable polymer may include poly(ester amide) (PEA), polyester, and poly(alkylene oxide), and combination thereof. Specific bioabsorbable polymers include PEA-40, polyethylene glycol (PEG), polypropylene glycol, poly(L-Lactide) (PLLA), poly(D-Lactide) (PDLA), poly(lactide-glycolide) (PLGA), poly(caprolactone) (PCL), block copolymer thereof, or blend thereof.

The microparticles or nanoparticles are partially or completely made of the bioabsorbable polymer mentioned above. The microparticles or nanoparticles may contain about 50% to 100%, for example, about 50%, about 60%, about 70%, about 80%, about 90%, or about 100% of the polymer mentioned above. The rest is made up by another biocompatible polymer suitable for making polymeric particles in combination with the polymer mentioned above, therapeutic agents, inorganic fillers, or combination thereof. The ratio of polymer to drug by weight may vary from 100:1 to 1:1, for example 90:1, 70:1, 50:1, 30:1, 10:1, 5:1, 3:1, and 2:1.

The microparticles or nanoparticles can be delivered through a catheter directly to the cancer tissue or to a feeding artery of the cancer tissue.

### Hydrogel

In some embodiments of the pharmaceutical composition, the carrier is a hydrogel. Preferably, the hydrogel is biodegradable.

In some embodiments, it is provided a method for the treatment of a liver cancer using hydrogel. The method comprises the following steps:

providing a composition that comprises a crosslinkable component,

providing a therapeutic agent in a pharmaceutically effective amount to the composition,

rendering the crosslinkable component crosslink to form a hydrogel, delivering the hydrogel containing the therapeutic agent to a blood vessel that directly services the diseased liver or the cancer tissue therein.

In some embodiments, the composition comprises an aqueous medium. In some embodiments, an aqueous medium is provided to the composition prior to activation of the crosslinking.

Various embodiments of crosslinkable components and means for rendering the crosslinkable component to crosslink, providing therapeutic agent, and delivering the pharmaceutical composition are described below. Hydrogels used in delivery of therapeutic agent can be formed outside of the body (ex vivo) or inside the body (in situ) of the subject. In some embodiments, the hydrogel is injectable and formed in situ. The injectable hydrogel comprises one or more polymer structures that are either inert or reactive with each other if activated. For reactive polymer structures, the composition can include an activation buffer or activation agent, i.e., a radical initiator. Reaction of the chemical structures (chemical crosslinking) can be induced by either the activation buffer or a radical initiator. The activation buffer or a radical initiator can be injected separately from the one or more of the polymer structures. Chemically crosslinked hydrogels can be prepared through photo-, thermal-, or pH activation to initiate chemical reactions such as reaction of thiols and acrylates, thiols and vinyls such as vinylsulfones, thiols and activated esters such as NHS(N-Hydroxy Succinimide)-esters, amines and activated esters, amines and vinyl/acrylates, thiols and thiols to from disulfide bonds, or any combination of the above. Physically crosslinked hydrogels can be formed by the self-assembly of polymers in response to environmental stimuli such as temperature, pH, solubility or a combination of those.

### Hydrogel Composition and Formation

In some embodiments, the hydrogel is a PEG/PEG in situ crosslinkable hydrogel. Preferably, the PEG/PEG in situ crosslinkable hydrogel are made from PEG/PEG polymers having multiple crosslinkable groups. Specific crosslinkable groups include thiol/NHS (N-hydroxy succinimide), thiol/acrylate, thiol/thiol, acrylate/acrylate, thiol/vinylsulfone, amine/NHS, and amine/aldehyde. As described herein, the crosslinkable groups in each pair are crosslinkable with each other, for example, thiol groups are crosslinkable with NHS groups. The crosslinking reaction of the in situ crosslinkable PEG/PEG is typically rapid and can be activated by a base or by free radical reactions initiated by peroxides, light, and/or temperature. Optionally, a crosslinker is used. Suitable crosslinkers include multi-functional polyethylene glycols (PEG), multifunctional PEG-PLGA copolymers, and multi-functional small molecules. The functionality can be thiols, amines, NHS-esters, acrylates, vinylsulfones, or aldehydes. The electrophilic groups (of number n) will react with the nucleophilic groups (of number m) and the total number of functional groups (m+n) should be 2+3, 2+4, 2+5 . . . 4+4, 4+5, 4+6, 4+7, 4+8 . . . 6+8, 7+8, 8+8 . . . 5+2, 4+2, 3+2 or always >4 in total. Thiols and acrylates can self-crosslink and any self-crosslinking system should have an average of more than 2 functional groups to gel meaning that some molecules could have at least two functional groups and some should have at least three functional groups.

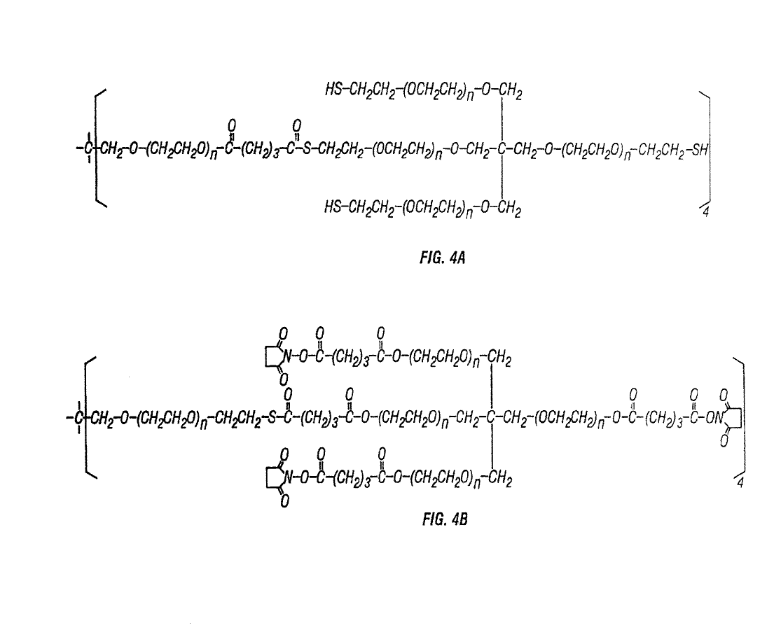

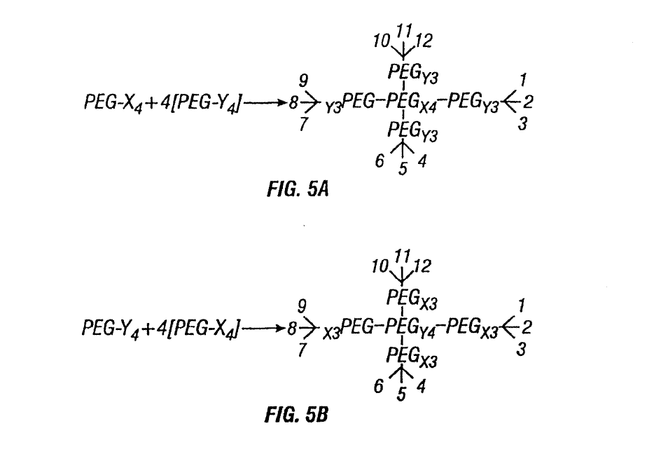

Multi-functionalized PEGs are of particular interest as crosslinkable PEG in the present invention. U.S. Pat. No. 6,534,591 to Rhee et al., U.S. Pat. No. 6,624,245 to Wallace et al., and U.S. Pat. No. 6,534,591 to Rhee et al. describe various multi-functionalized polymers especially PEGs that can be used to form hydrogel, the teachings of which are incorporated by reference herein. Multi-functionalized PEGs refer to PEGs that bear at least two functional groups per molecule, for example, three (tri-functional or tri-functionalized), four (tetra-functional or functionalized), six (hepta-functional or functionalized), eight (octa-functional or functionalized), and so on. FIG. 4 depicts exemplary tetra-functionalized PEGs and FIG. 5 depicts exemplary 4+4 intermediates formed by multi-functionalized PEGs. Any combination of functionality is also possible, such as 4+6, or 3+8 are also possible.

In various embodiments of the present invention, the composition for making crosslinkable PEG/PEG hydrogel comprise (a) a first crosslinkable component having m nucleophilic groups, wherein m≧2; and (b) a second crosslinkable component having n electrophilic groups capable of reaction with the m nucleophilic groups to form covalent bonds, wherein n≧3, and m+n≧5.

Examples of such nucleophilic groups include primary amines, thiols, and hydroxyl groups. Examples of such electrophilic groups include acid chloride groups, anhydrides, activated esters, ketones, aldehydes, isocyanate, isothiocyanate, epoxides, and olefins, including conjugated olefins such as vinylsulfone, acrylates, maleimides and analogous functional groups. Typical in situ crosslinking reactions include reaction of an amine and a NHS to form an amide, reaction of an aldehyde and an amine to form a Schiff base, reaction of an aldehyde and hydrazide to form a hydrozone, and Michael reaction of an acrylate and either a primary amine or a thiol to form a secondary amine or a sulfide.

The composition may be administered before, during or after the components inter-react in the aqueous environment to form a three-dimensional matrix.

The composition of the present invention is generally delivered to the site of administration in such a way that the individual reactive groups of the compounds are exposed to the aqueous environment for the first time at the site of administration, or immediately preceding administration. Thus, the composition is preferably delivered to the site of administration using an apparatus that allows the composition to be delivered in dry environment, where the compounds are essentially non-reactive.

In some embodiments, a three-dimensional matrix is formed by the steps of: (a) providing a composition described above; (b) rendering the nucleophilic and electrophilic groups reactive by exposing the composition to an aqueous environment to effect inter-reaction; wherein said exposure comprises: (i) dissolving the composition in a first buffer solution having a pH within the range of about 1.0 to 5.5 to form a homogeneous solution, and (ii) adding a second buffer solution having a pH within the range of about 6.0 to 11.0 to the homogeneous solution; and (c) allowing a three-dimensional matrix to form. Typically, the matrix is formed, e.g., by polymerization, without input of any external energy.

The first and second components of the composition are typically combined in amounts such that the number of nucleophilic groups in the mixture is approximately equal to the number of electrophilic groups in the mixture. As used in this context, the term “approximately” refers to a 2:1 to 1:2 ratio of moles of nucleophilic groups to moles of electrophilic groups. A 1:1 molar ratio of nucleophilic to electrophilic groups is generally preferred.

The first and second components are blended together to form a homogeneous dry powder. This powder is then combined with a buffer solution having a pH within the range of about 1.0 to 5.5 to form a homogeneous acidic aqueous solution, and this solution is then combined with a buffer solution having a pH within the range of about 6.0 to 11.0 to form a reactive solution.

In some embodiments, the composition for making crosslinkable PEG/PEG hydrogel comprises one or more crosslinkable components that are self-crosslinkable and having multiple self-crosslinkable functional groups such as acrylic functional groups or thiols. The crosslinking can be activated by irradiation and/or a radical initiator.

### Incorporation of Therapeutic Agents

Therapeutic agents, including small molecule drugs and biologics, can be incorporated or loaded into the hydrogel in various ways. In some embodiments, the therapeutic agent is loaded through encapsulation or entrapment wherein the therapeutic agent is encapsulated during the network crosslinking. Typically, this is done by admixing the gel forming polymer(s) with the therapeutic agent.

In some embodiments, the therapeutic agent is loaded through tethering wherein the therapeutic agent is covalently attached to the hydrogel directly or via a linker. The bond between the therapeutic agent and the hydrogel or the linker is degradable by enzyme or hydrolysis. U.S. Pat. No. 5,162,430 to Rhee et al. describes processes for covalent attaching biologically active agents to the functional groups on synthetic polymers, the teaching of which is incorporated herein by reference.

In some embodiments, the therapeutic agent can be physically attached to the hydrogel via a physical force such as hydrogen bonding, negative-positive charge interaction, and hydrophobic interaction.

In some embodiments, the therapeutic agent is loaded through a polymeric carrier. For example, the therapeutic agent is loaded through incorporation of polymeric microparticles or nanoparticles that are embedded or impregnated with the therapeutic agent. In this method, the therapeutic agent is first embedded or impregnated into nanoparticles or microparticles and then the particles are entrapped or encapsulated in a hydrogel polymer network. This method is particularly useful and advantageous for delivery of therapeutic agent that is reactive to the functional groups in crosslinkable components or is sensitive to the pH of the buffers. The particles protect these therapeutic agents from being reacted by or otherwise rendered inactive by the crosslinkable components and the crosslinking environment. Also, these particles can function as a carrier for hydrophobic drugs such as paclitaxel, zotarolimus, etc. which is hardly soluble in the aqueous solution for hydrogel formation. Additionally, these particles can be used for control release of therapeutic agents which is either highly hydrophilic or substantially smaller than the pore size of the hydrogel which may have undesirable burst effect if loaded alone. The sizes of the particles and the polymers that can be used to make the particles include those described in a previous section of the specification.

For delivery of biologics that have high molecular weight and large size, the crosslinkable components can be made more biodegradable so that the biologics can be released upon dissolution of the hydrogel as well as diffusion from the hydrogel network.

In some embodiments, when radical initiator is used or free radical is generated in crosslinking, a free radical scavenger can be added to the crosslinking composition to prevent damage of the therapeutic agent by the free radical as necessary. An exemplary free radical scavenger is (2,2,6,6-tetramethylpiperidin-1-yl)oxidanyl (TEMPO).

In some embodiments, the therapeutic agent is loaded through incubating the hydrogel in concentrated therapeutic agent solution. This method is particularly suitable for incorporating therapeutic agent to a hydrogel that is formed ex vivo.

In some embodiments of the present invention, the anti-angiogenesis agent, e.g., anti-VEGF antibody (e.g., bevacizumab) or anti-EGRF (e.g., ABT-806), is loaded into a PEG/PEG crosslinkable hydrogel by entrapment. In some embodiments, the agent is loaded into a PEG/PEG crosslinkable hydrogel through polymeric microparticles or nanoparticles.

In some embodiments of the present invention, the anti-angiogenesis agent, e.g., ABT-896, sorafenib, or ABT-348 is loaded into a PEG/PEG crosslinkable hydrogel by entrapment. In some embodiments, the agent is loaded into a PEG/PEG crosslinkable hydrogel through polymeric microparticles or nanoparticles.

In some embodiments where two or more agents are loaded into a hydrogel, the agents can be loaded in the same way or in different ways. For example, when two agents are loaded, the first agent is incorporated into polymeric particles and then the particles and the second agent are loaded into a hydrogel by entrapment.

Delivery of Therapeutic Agent to a Tumor Tissue Via Hydrogel

The delivery of hydrogel loaded with therapeutic agent(s) can be achieved by using a catheter, a needle, or a syringe. In some embodiments, the hydrogel is delivered through a catheter to the feeding artery proximal to a tumor tissue. In some embodiments, the hydrogel is delivered through a catheter directly to the tumor tissue. In some embodiments, the hydrogel can be injected by a needle balloon catheter peritumorally (around the tumor tissue) or intralesionally (within the tumor tissue) under X-ray guidance. In various embodiments, the tumor tissue is a liver cancer tissue.

In some embodiments, the hydrogel is delivered by a multi-compartment device. US 2012/0041481 by Daniloff et al. describes multi-compartment delivery devices that can be used for hydrogel delivery, the teaching of which is incorporated herein by reference.

In the present invention, suitable delivery systems for the homogeneous dry powder composition and the two buffer solutions describe above may involve a multi-compartment device, where one or more compartments contain the powder and one or more compartments contain the buffer solutions needed to provide for the aqueous environment, so that the composition is exposed to the aqueous environment as it leaves the compartment. Alternatively, the composition can be delivered using any type of controllable extrusion system, or it can be delivered manually in the form of a dry powder, and exposed to the aqueous environment at the site of administration.

The homogeneous dry powder composition and the two buffer solutions may be conveniently formed under aseptic conditions by placing each of the three ingredients (dry powder, acidic buffer solution and basic buffer solution) into separate syringe barrels. For example, the composition, first buffer solution and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

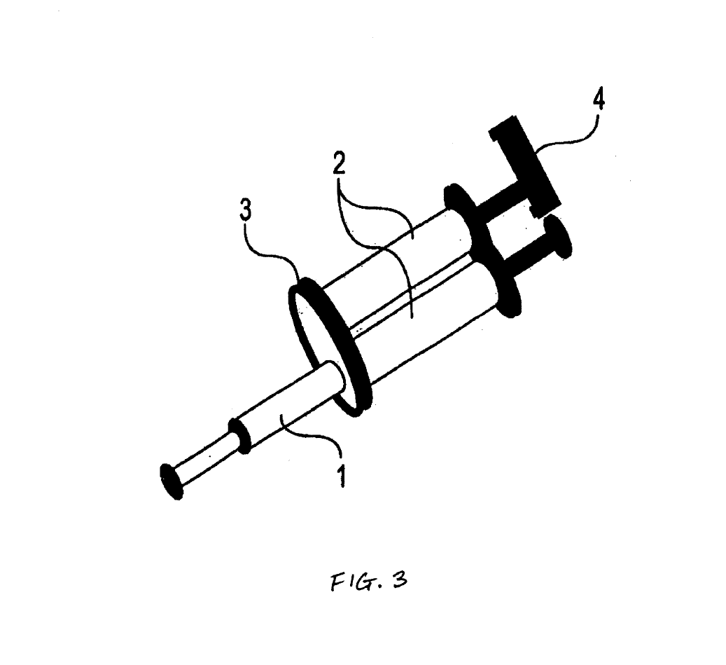

An exemplary multi-compartment syringe system of the present invention is shown in FIG. 3. The device is comprised of three syringes, two housing each of the two buffers of the present invention with the third syringe housing the dry powder composition 1. The two syringes housing the solutions 1 are pre-assembled into a syringe housing 2 with a transfer port closure 3 attached to the housing assembly 2 to allow mixing of the dry powder into the correct syringe. A syringe clip 4 is attached to the plunger rod of the syringe that does not require mixing with the dry powder composition.

A multi-compartment catheter system can be used to deliver hydrogel to a feeding artery to the tumor tissue or tumor tissue itself in the present invention.

In some embodiments, the pharmaceutical composition comprises a hydrogel and preferably an anti-VEGF antibody or anti-EGRF anti-body. In some embodiments, the pharmaceutical composition further comprises embolic beads in the hydrogel. In some embodiments, the embolic beads are embedded or impregnated with a radioactive isotope, a radioactive anti-cancer drug, a biologic, or a chemotherapy drug.

In some embodiments, embolic beads are delivered to plug the distal arterial bed of the artery prior to delivering the hydrogel to the artery.

The second therapeutic agent can be embedded or impregnated in the microparticles or nanoparticles or can be dispersed in the hydrogel.

In some embodiments, the local delivery of therapeutic agent is combined with a systemic delivery of therapeutic agent, wherein the two modes of delivery are additive or synergic to each other. Exemplary systemic delivery includes oral administration and intravenous injection or infusion.

The use of biodegradable hydrogels has a number of advantages. For example, hydrogels have high hydrophilicity and therefore high biocompatibility. The properties such as gelation time, network pore size, chemical functionalization, and degradation time of hydrogels can be made suitable for desired applications.

### Exemplary Bioresorbable Polymer Scaffolds

Any bioresorbable polymer scaffold that can be inserted into a site, such as the lumen of a blood vessel connected to a diseased liver, can be used to in the present invention. In some embodiments, the bioresorbable polymer scaffold is an implantable device, such as a stent. A stent will be used as an example to illustrate the characteristics of an exemplary bioresorbable polymer scaffold. However, one of skill in the art would understand that any device made of bioresorbable polymer that is suitable for delivering one or more therapeutic agents to a diseased liver can be used in the present invention.

Stents are generally cylindrically shaped devices that function to hold open and sometimes expand a segment of a blood vessel or other anatomical lumen such as urinary tracts and bile ducts. Stents are often used in the treatment of atherosclerotic stenosis in blood vessels.

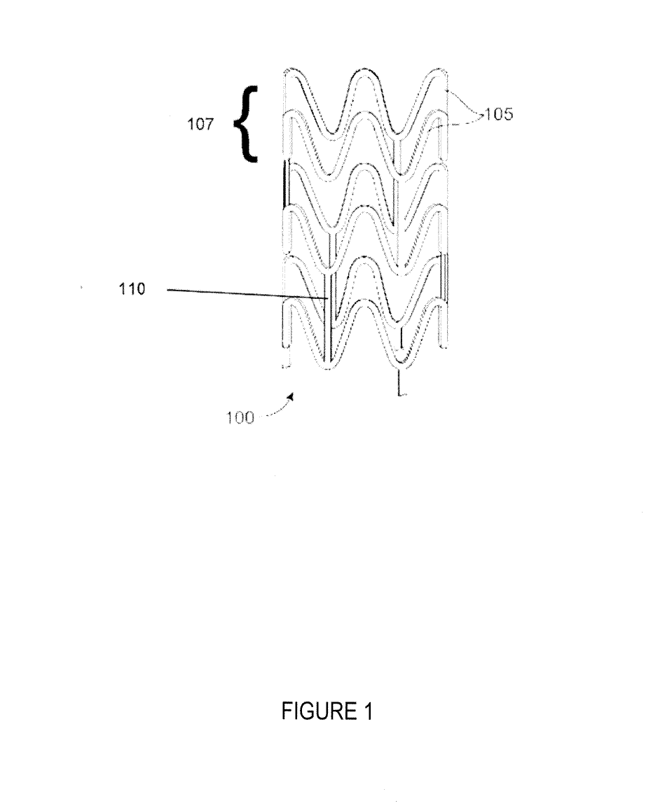

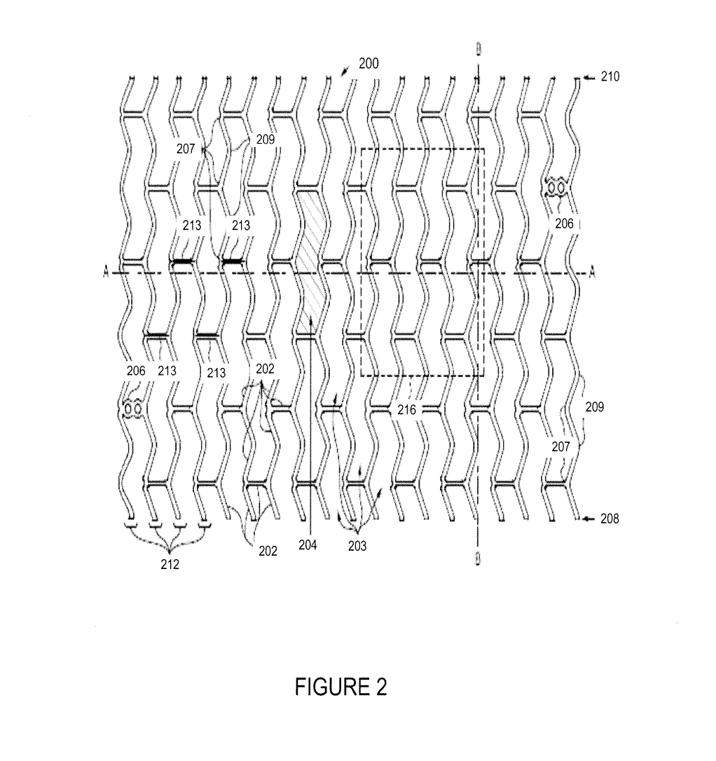

Stents are typically composed of a scaffold or scaffolding that includes a pattern or network of interconnecting structural elements or struts, formed from wires, tubes, or sheets of material rolled into a cylindrical shape (see, for example, FIGS. 1 and 2). This scaffolding gets its name because it physically holds open and, if desired, expands the wall of the passageway. Typically, stents are capable of being compressed or crimped onto a catheter so that they can be delivered to and deployed at a treatment site.

FIG. 1 depicts a view of an exemplary stent 100. In some embodiments, a stent may include a body, substrate, or scaffold having a pattern or network of interconnecting structural elements 105. Stent 100 may be formed from a tube (not shown). FIG. 1 illustrates features that are typical to many stent patterns including undulating sinusoidal cylindrical rings 107 connected by linking elements 110. As mentioned above, the cylindrical rings are load bearing in that they provide radially directed force to support the walls of a vessel. The linking elements generally function to hold the cylindrical rings together. A structure such as stent 100 having a plurality of structural elements may be referred to as a stent scaffold or scaffold. Although the scaffold may further include a coating, it is the scaffolding structure that is the load bearing structure that is responsible for supporting lumen walls once the scaffolding is expanded in a lumen.

The structural pattern in FIG. 1 is merely exemplary and serves to illustrate the basic structure and features of a stent pattern. A stent such as stent 100 may be fabricated from a polymeric tube or a sheet by rolling and bonding the sheet to form the tube. A tube or sheet can be formed by extrusion or injection molding. A stent pattern, such as the one pictured in FIG. 1, can be formed on a tube or sheet with a technique such as laser cutting or chemical etching. The stent can then be crimped on to a balloon or catheter for delivery into a bodily lumen.

Alternatively, the scaffold design may be composed of radial bands that slide to increase the diameter of the scaffold. Such a design utilizes a locking mechanism to fix the stent at a target diameter and to achieve final radial strength. In other embodiments, the scaffold design could be braided polymer filaments or fibers.

In a preferred embodiment a stent scaffold has the stent pattern described in U.S. Patent Publication No. US 2010/0004735 by Yang et al. Other examples of stent patterns suitable for PLLA are found in US Patent Publication No. 2008/0275537. FIG. 2 depicts exemplary stent pattern 200 from US 2008/0275537. The stent pattern 200 is shown in a planar or flattened view for ease of illustration and clarity, although the stent pattern 200 on a stent actually extends around the stent so that line A-A is parallel or substantially parallel to the central axis of the stent. The pattern 200 is illustrated with a bottom edge 208 and a top edge 210. On a stent, the bottom edge 208 meets the top edge 210 so that line B-B forms a circle around the stent. In this way, the stent pattern 200 forms sinusoidal hoops or rings 212 that include a group of struts arranged circumferentially. The rings 212 include a series of crests 207 and troughs 209 that alternate with each other. The sinusoidal variation of the rings 212 occurs primarily in the axial direction, not in the radial direction. That is, all points on the outer surface of each ring 212 are at the same or substantially the same radial distance away from the central axis of the stent.

The stent pattern 200 includes various struts 202 oriented in different directions and gaps 203 between the struts. Each gap 203 and the struts 202 immediately surrounding the gap 203 define a closed cell 204. At the proximal and distal ends of the stent, a strut 206 includes depressions, blind holes, or through holes adapted to hold a radiopaque marker that allows the position of the stent inside of a patient to be determined.

One of the cells 204 is shown with cross-hatch lines to illustrate the shape and size of the cells. In the illustrated embodiment, all the cells 204 have the same size and shape. In other embodiments, the cells 204 may vary in shape and size.

Still referring to FIG. 2, the rings 212 are connected to each other by another group of struts that have individual lengthwise axes 213 parallel or substantially parallel to line A-A. The rings 212 are capable of being collapsed to a smaller diameter during crimping and expanded to their original diameter or to a larger diameter during deployment in a vessel. Specifically, pattern 200 includes a plurality of hinge elements. When the diameter of a stent having stent patter 200 is reduced or crimped, the angles at the hinge elements decrease which allow the diameter to decrease. The decrease in the angles results in a decrease in the surface area of the gaps 203.

In some embodiments, the stent scaffold has a stent pattern described in U.S. Patent Application Publication No. 2011/0190872 by Anukhin et al.

Dimensions of the stent for hepatic applications depend upon such factors as the size of the anatomical lumen that is to be treated. For example, the diameter of the scaffold is 2 to 8 mm, 4 to 7 mm, 3 to 5 mm, or more narrowly 2.5 to 3.5 mm. In some embodiments, bioabsorbable polymer scaffold of smaller diameters (e.g., less than 2 mm) or larger diameters (e.g., more than 10 mm) may be used. In general the length of the scaffold is 8 to 38 mm, or more narrowly, 8 to 12 mm, 12 to 18 mm, 15 to 18 mm, 18 to 24 mm, 18 to 38 mm. In preferred embodiments, a bioabsorbable polymer scaffold has a diameter of 4-7 mm. In preferred embodiments, a bioabsorbable polymer scaffold has a length at 12 mm, 15 mm or 18 mm. All diameter ranges refer to inner or outer diameter and the as-fabricated or deployed diameter. The scaffolds for hepatic treatment have sufficient radial strength to support the vessels at a target diameter.

In the present invention, a stent is used primarily for drug delivery. In certain embodiments, the radial strength required for the present invention may be enough to secure the stent (or a similar device) at the desired locale for drug delivery purposes without expanding or significantly expanding the size of the locale or site where the stent is placed (e.g., a hepatic artery). In some embodiments, the locale or site remains its original size. In some embodiments, the diameter of the locale or site is only slightly greater than its original size in order to secure the stent; for example, by about 15% or less, 12% or less, 10% or less, 8% or less, 5% or less, 3% or less, 2% or less, 1% or less, between 1 and 15%, between 2 and 12%, between 5 and 10%.

Stents fabricated from bioresorbable, biodegradable, bioabsorbable, and/or bioerodable materials such as bioabsorbable polymers can be designed to completely absorb only after or some time after the clinical need for them has ended. Consequently, a fully bioabsorbable stent can reduce or eliminate the risk of potential long-term complications and of late thrombosis and facilitate non-invasive diagnostic MRI/CT imaging.

The use of bioabsorbable polymer stents has a number of advantages. (i) The stent disappears from the treated site resulting in reduction or elimination of late stent thrombosis. (ii) Disappearance of the stent facilitates repeat treatments (surgical or percutaneous) to the same site. (iii) Disappearance of the stent allows restoration of vasomotion at the treatment site. (iv) The bioabsorbability results in freedom from side-branch obstruction by struts.

### Delivering Therapeutic Agents to Diseased Liver Via Bioresorbable Polymer Scaffold

In one aspect of the invention, a therapeutic agent is delivered by a bioresorbable polymer scaffold. In some embodiments, the bioresorbable polymer scaffold comprises a polymer substrate and a coating comprising a polymer matrix. In some embodiments, the coating comprises one or more layers in any combination, including but not limited to a primer layer, a reservoir layer, a topcoat layer, or a biocompatible finishing layer, which may improve the biocompatibility of the coating.

In some embodiments, the polymer matrix is made of an amorphous polymer or an amorphous mixed of polymers. In some embodiments, the polymer substrate is made of a crystalline form of polymer or crystalline form of mixed of polymers. In some embodiments, the bioabsorbable polymer scaffold comprises only a polymer substrate without a polymer matrix coating.

In some embodiments, one or more therapeutic agents are embedded or impregnated in the polymer substrate and the polymer matrix. In some embodiments, one or more therapeutic agents are embedded or impregnated only in the polymer matrix. The scaffold may be free of therapeutic agent or a particular type of therapeutic agent other than incidental diffusion of agent into the scaffold from the polymer matrix. In some embodiments, one or more therapeutic agents are embedded or impregnated only in the polymer substrate. The polymer matrix may be free of therapeutic agent or a particular type of therapeutic agent other than incidental diffusion of agent into the scaffold from the scaffold. In some embodiments, the polymer matrix is absent from the bioabsorbable polymer scaffold, and one or more therapeutic agents are embedded or impregnated in the polymer substrate alone.

The therapeutic agent may be released from the bioresorbable scaffold by diffusion from the polymer or by erosion of the polymer. In some embodiments, a therapeutic agent is delivered to the site of action (e.g., a lumen of a blood vessel that is connected to a diseased liver) from both the polymer matrix and polymer substrate of the bioresorbable polymer scaffold. In some embodiments, the therapeutic agent is delivered to the site of action in a two-stage process in which the therapeutic agent is released from the polymer matrix and polymer substrate at different rates.

In some embodiments, the polymer matrix (such as a coating) is a thin coating layer that comprises an amorphous (non-crystalline) polymer such as poly(DL-lactide) (PDLLA). In some embodiments, the polymer matrix comprises a therapeutic agent. The ratio of the therapeutic agent (e.g., a small molecule therapeutic agent): polymer matrix (e.g., PDLLA) may vary, for example, is about 5:1, about 4:1, about 3:1, about 2:1, about 1:1, about 1:2, about 1:3, about 1:4, about 1:5. Preferably, the ratio is about 1:1. In some embodiments, the coating has a thickness of less than about 10 μm, between about 10 and about 20 μm, between about 20 and about 30 lam, between about 20 and about 40 μm, between about 10 and about 40 μm, between about 10 and about 50 μm, between about 40 and about 50 μm, or over about 50 μm. Preferably, the thickness is about 30 to about 50 μm. In an exemplary embodiment, amorphous PDLLA and a small molecule therapeutic agent (at a ratio of about 1:1) are combined to form a matrix coating layer that is between 30 and 50 μm. The loading of the therapeutic agent is between about 0.5 mg/cm² and about 5 mg/cm², for example, between about 1 mg/cm² and about ⁵ mg/cm², or between about 1 mg/cm² and about 4 mg/cm², preferably between about 1 mg/cm² and about 3 mg/cm². The coating releases the therapeutic agent in a time-controlled manner over an extended period of time.

In some embodiments, polymers forming the substrate of the bioresorbable polymer scaffold are highly crystalline such that it provides structure integrity to the bioresorbable polymer scaffold. In some embodiments, the polymers used to form the substrate comprise poly(L-lactide) (PLLA). In some embodiments, the polymer substrate also comprises a therapeutic agent. The crystallinity of the polymer forming the polymer substrate is between about 20% and 60%, for example, between about 30% and 60%, between about 40% and 60%, between about 40% and 50%, or between about 35% and 45%. In an exemplary embodiment, crystalline PLLA and a small molecule therapeutic agent are combined to form the polymer substrate. The bioresorbable polymer scaffold is processed for increased radial strength. The thickness of the substrate varies between about 50 μm and about 500 μm, preferably between 100 μm and 200 μm.

In some embodiments, the therapeutic agent in the polymer substrate is the same as the one in the polymer matrix coating. In some embodiments, the therapeutic agent in the polymer substrate is different from the one in the polymer matrix coating. In some embodiments, the polymer substrate comprises more than one therapeutic agent. In some embodiments, the polymer matrix coating also comprises more than one therapeutic agent. In some embodiments, the polymer matrix coating and polymer substrate share at least one common therapeutic agent. In some embodiments, the polymer matrix coating and polymer substrate do not share at least one common therapeutic agent.

In some embodiments, a therapeutic agent is released from the polymer matrix and polymer substrate at the same time with the substrate having a longer lasting release profile, but at different rates. In some embodiments, a therapeutic agent is released from the polymer matrix and the polymer substrate at the same time at similar rate. In some embodiments, a therapeutic agent is released from the polymer matrix and the polymer substrate at a different rate, for example, release from the polymer matrix has a shorter-release profile due to, for example, the smaller dimension of the polymer matrix (e.g., a thin coating) and release from the substrate has a longer lasting release profile due to, for example, the larger dimension of the polymer substrate.

In some embodiments, the therapeutic agent is delivered to the site of action in a two-stage process in which the therapeutic agent is released from the polymer matrix and polymer substrate in an overlapping manner or nearly sequential manner. In some embodiments, the polymer matrix is partially or completely absorbed before the polymer substrate started to be absorbed.

### Additional Characteristics of Bioresorbable Polymer Scaffolds

Bioresorbable polymer scaffolds include, but are not limited to, self-expandable stents, balloon-expandable stents, stent-grafts, and generally tubular medical devices in the treatment of liver cancers. The present invention is further applicable to various stent designs including wire structures, and woven mesh structures.

Self-expandable or self-expanding stents include a bioabsorbable polymer scaffold that expands to the target diameter upon removal of an external constraint without assistance of a radial outward force. However, self-expandable stents can be assisted by such a radial outward force. The self expanding scaffold returns to a baseline configuration (diameter) when an external constraint is removed. This external constraint could be applied with a sheath that is oriented over a compressed scaffold. The sheath is applied to the scaffold after the scaffold has been compressed by a crimping process. After the stent is positioned at the implant site, the sheath may be retracted by a mechanism that is available at the end of the catheter system and is operable by the physician. The self expanding bioabsorbable scaffold property is achieved by imposing only elastic deformation to the scaffold during the manufacturing step that compresses the scaffold into the sheath.

The bioresorbable scaffold may also be expanded by a balloon. In this embodiment the scaffold is plastically deformed during the manufacturing process to tightly compress the scaffold onto a balloon counted on a catheter system. The scaffold is deployed at the treatment site by inflation of the balloon. The balloon will induce areas of plastic stress in the bioabsorbable material to cause the scaffold to achieve and maintain the appropriate diameter on deployment.

The prevailing mechanism of degradation of many bioabsorbable polymers is chemical hydrolysis of the hydrolytically unstable substrate. In a bulk degrading polymer, the polymer is chemically degraded throughout the entire polymer volume. As the polymer degrades, the molecular weight decreases. The reduction in molecular weight results in changes in mechanical properties (e.g., strength) and stent properties. For example, the strength of the scaffold material and the radial strength of the scaffold is maintained for a period of time followed by an gradual or abrupt decrease. The decrease in radial strength is followed by a loss of mechanical integrity and then erosion or mass loss. Mechanical integrity loss is demonstrated by cracking and by fragmentation. Enzymatic attack and metabolization of the fragments occurs, resulting in a rapid loss of polymer mass.

In embodiments of the present invention, the bioresorption properties of scaffolds are adjusted for treatment of liver cancers. The scaffold biodegradation properties such as the resorption time and the support time are adjusted depending on the clinical need for various conditions. The support time may be dictated by one or more considerations, depending on the treatment, such as time needed for therapeutic agent to be released into the diseased liver, for example, into the region of where the liver tumor is located.

The manufacturing process of a bioabsorbable scaffold includes selection of a bioabsorbable polymer raw material or resin. Detailed discussion of the manufacturing process of a bioabsorbable stent can be found elsewhere, e.g., U.S. Patent Publication No. 20070283552. The fabrication methods of a bioabsorbable stent can include the following steps:

(1) forming a polymeric tube from a biodegradable polymer resin using extrusion,

(2) optionally radially deforming the formed tube to increase radial strength,

(3) forming a stent scaffolding from the deformed tube by laser machining a stent pattern in the deformed tube with laser cutting, in exemplary embodiments, the strut thickness can be 100-200 microns, or more narrowly, 120-180, 130-170, or 140-160 microns,

(4) optionally forming a therapeutic coating over the scaffolding,

(5) crimping the stent over a delivery balloon, and

(6) sterilization with election-beam (E-beam) radiation.

Poly(L-lactide) (PLLA) is attractive as a stent for applications in which a vessel diameter requires maintaining patency (e.g., as the substrate or scaffold material) due to its relatively high strength and a rigidity at human body temperature, about 37° C. Since it has a glass transition temperature between about 60 and 65° C. (Medical Plastics and Biomaterials Magazine, March 1998), it remains stiff and rigid at human body temperature. This property facilitates the ability of a PLLA stent scaffold to maintain a lumen at or near a deployed diameter without significant recoil (e.g., less than 10%). In general, the Tg of a semi-crystalline polymer can depend on its morphology, and thus how it has been processed. Therefore, Tg refers to the Tg at it relevant state, e.g., Tg of a PLLA resin, extruded tube, expanded tube, and scaffold.

Additional exemplary biodegradable polymers for use with a bioresorbable polymer scaffolding include poly(D-lactide) (PDLA), polymandelide (PM), polyglycolide (PGA), poly(L-lactide-co-D,L-lactide) (PLDLA), poly(D,L-lactide) (PDLLA), poly(D,L-lactide-co-glycolide) (PLGA) and poly(L-lactide-co-glycolide) (PLLGA). With respect to PLLGA, the stent scaffolding can be made from PLLGA with a mole % of GA between 5 and 15 mol %. The PLLGA can have a mole % of (LA:GA) of 85:15 (or a range of 82:18 to 88:12), 95:5 (or a range of 93:7 to 97:3), or commercially available PLLGA products identified as being 85:15 or 95:5 PLLGA. The examples provided above are not the only polymers that may be used.

Polymers that are more flexible or that have a lower modulus that those mentioned above may also be used. Exemplary lower modulus bioabsorbable polymers include, polycaprolactone (PCL), poly(trimethylene carbonate) (PTMC), polydioxanone (PDO), poly(4-hydroxy butyrate) (PHB), and poly(butylene succinate) (PBS), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of a blend can include 1-5 wt % of low modulus polymer.

An exemplary PLLA scaffold may have an initial L-lactide monomer content within the range of less than 0.02 wt %, 0.02 to 0.2 wt %, and 0.02 wt % to 1 wt %, or any sub-range or value in these ranges. The Mn(0) (molecular weight at the time of implantation) of PLLA can be at least 60 kDa, 60 to 66 kDa, 66 to 80 kDa, 80 to 120 kDa, greater than 120 kDa or any sub-range or value in these ranges. An exemplary PLLA scaffold can have any combination of these Mn(0) and L-lactide monomer content.

The term “molecular weight” can refer to one or more definitions of molecular weight. “Molecular weight” can refer to the molecular weight of individual segments, blocks, or polymer chains. “Molecular weight” can also refer to weight average molecular weight or number average molecular weight of types of segments, blocks, or polymer chains.

In some embodiments, the scaffold deployed completely absorbs away in less than 1 year, less than 2 years, between 1 and 2 years, between 1.5 and 2 years, between 2 and 2.5 years, or greater than 2.5 years. The support time and the resorption time of a scaffold can be adjusted through initial molecular weight of the scaffold material, monomer content of the scaffold material, or both. For example, the scaffold material is PLLA and the LLA monomer content is adjusted.

The target diameters range of the deployed scaffolds, which can correspond, but not necessarily, to the diameters of the scaffolds as fabricated before crimping. The target diameter can be between 2 and 8 mm, or more narrowly 2 to 5 mm. The target diameter can be based on a diameter of the lumen in which the scaffold is to be deployed.

The length of the scaffolds can be between about 4 mm and about 40 mm. When multiple scaffolds are used, the lengths of the scaffolds can be the same or different. The length of the scaffold can be tailored.

In some embodiments, a bioresorbable polymer scaffold can be introduced into surgically created cavity with any area of the liver where a tumor is located. In these embodiments, bioresorbable polymer scaffolds deliver therapeutic agents directly to the diseased area within the diseased liver in order to achieve high efficiency.

The method of treatment may further include implanting at least one additional bioresorbable scaffold at the site of deployment of at least one of the scaffolds after it has partially or completely absorbed. The additional scaffold may be deployed at a greater diameter than the initial scaffold to accommodate for drug delivery to a diseased liver.

### Additional Characteristics of Hydrogel

Nguyen and Lee (Macromol. Biosci. 2010, 10, 563-579) discloses a series of temperature or pH-temperature sensitive polymers that can be used to prepare hydrogels via physical crosslinking Polymers that are sensitive to temperature, pH, or both and can be used to prepare physically crosslinked hydrogel include poly(ethyleneglycol)(PEG)/polyester block copolymers, polyphosphazenes, polypeptides, chitosan, polymers based on sulfamethazine, poly(β-aminoester) (PAE), poly(aminourethane) (PAU), poly(amidoamine) (PAA), and others.

In the present invention, the hydrogel can be prepared from inert polymers that are temperature or pH-temperature sensitive. In some embodiments, the temperature sensitive polymer is a PEG-PLLA-PEG triblock copolymer, a PEG-PDLA-PEG triblock copolymer or PDLA-PEG-PDLA triblock copolymer, a PEG-PEG-PLGA-PEG triblock copolymer or PLGA-PEG-PLGA triblock copolymer, a PCL-PEG-PCL copolymer or PEG-PCL-PEG copolymer, a PCTC-PEG-PCTC copolymer or PEG-PCTC-PEG copolymer, a multiblock copolymer consisting of PEG, PPG, and PHB.

In some embodiments, the pH-temperature sensitive polymer is OSM-PCLA-PEG-PCLA-OSM or OSM-PCGA-PEG-PCGA-OSM, or PAE-PCL-PEG-PCL-PAE pentablock copolymer, PCL-PEG-PCL-PAU, (PEG-PAU)m, PAA-PEG-PAA, and PAE-PEG-PAE.

Here PCTC stands for to poly(caprolactone-co-trimethylene carbonate), PCL stands for poly(caprolactone), PCLA stands for poly(caprolactone-co-lactide), PCGA stands for poly(caprolactone-co-glycotide), PPG stands for poly(propylene glycol), PHB stands for poly(3-hydroxybutyrate), OSM stands for acidic sulfamethazine oligomers.

Hydrogel prepared via physical crosslinking has certain advantages. For example, they are inert so that undesired chemical reaction with the therapeutic agent incorporated therein can be avoided.

### EXAMPLES

### Example 1

### Delivery of an Anti-Angiogenesis Antibody Via Hydrogel

An injectable composition comprising an in situ crosslinkable PEG/PEG mixed with an anti-VEGF monoclonal antibody (e.g., Avastin) or an anti-EGRF monoclonal antibody (e.g., ABT806) is prepared. Examples of the PEG/PEG crosslinkable hydrogels include those having crosslinkable groups such as thiol/NHS, thiol/acrylate, thiol/thiol, acrylate/acrylate, thiol/vinylsulfone, amine/NHS, and amine/aldehyde. The crosslinking reaction is rapid and activated by base or by free radical reactions initiated by peroxides, light, and/or temperature.

The composition is delivered trans-arterially through a catheter to the feeding artery proximal to a tumor tissue. The crosslinking reaction is initiated in situ and a hydrogel forms in situ.

### Example 2

### Delivery of an Anti-Angiogenesis Small Molecule Therapeutic Agent Via Hydrogel

An injectable composition comprising an in situ crosslinkable PEG/PEG mixed with a small molecule therapeutic agent (e.g., ABT-869 or sorafenib) is prepared. Examples of PEG/PEG crosslinkable hydrogels include those having cross-linkable groups such as thiol/NHS, thiol/acrylate, thiol/thiol, acrylate/acrylate, thiol/vinylsulfone, amine/NHS, and amine/aldehyde. The crosslinking reaction is rapid and activated by base or by free radical reactions initiated by peroxides, light, and/or temperature.

The composition is delivered trans-arterially through a catheter to the feeding artery proximal to a tumor tissue. The crosslinking reaction is initiated in situ and a hydrogel forms in situ.

### Example 3

### Delivery of Small Molecule Anti-Angiogenesis Agent Via Microparticles or Nanoparticles

Microparticles or nanoparticles containing small molecule therapeutic agent such as sorafenib are delivered trans-arterially through a catheter directly to a tumor tissue.

The particles can be made of poly(ester amide)s or polyesters, particularly PEA-40, PLLA, PDLA, PLGA, PCL, PEG, block copolymers thereof, or block copolymers of these polymers with PEG.

### Example 4

### Delivery of Small Molecule Anti-Angiogenesis Agent Via Microparticles or Nanoparticles in Hydrogel

Bioabsorbable polymeric microparticles or nanoparticles containing a small molecule therapeutic agent such as sorafenib are provided. The particles are mixed with a PEG/PEG in-situ crosslinkable hydrogel. The hydrogel is delivered trans-arterially through a catheter to the feeding artery proximal to a tumor tissue.

Examples of PEG/PEG crosslinkable hydrogels include those having crosslinkable groups such as thiol/NHS, thiol/acrylate, thiol/thiol, acrylate/acrylate, thiol/vinylsulfone, amine/NHS, and amine/aldehyde. The crosslinking reaction can be rapid and activated by base or by free radical reactions initiated by peroxides, light, and/or temperature.

### Example 5

### Delivery of Small Molecule Anti-Angiogenesis Agent Via a Drug Eluting Scaffold

Soafenib is embedded or impregnated in a bioabsorbable polymer stent body or a bioabsorbable polymer coating of a stent. The stent is deployed to an artery proximal to a tumor tissue. Soafenib is released from the stent by diffusion or erosion.

### Example 6

### Exemplary Embodiments

### 6A. Local or Targeted (Site-Specific or Regional) Drug Delivery

This method allows for better bioavailability, low systemic toxicity, and use of drugs that are hard to formulate for systemic delivery.

### 6A1. Local Delivery of Small Molecule Therapeutic Agent Via Bioabsorbable Scaffold:

A bioabsorbable scaffold is provided according to the following specification:

| Drug loading: | 1 | mg/cm² |
|---|---|---|
| Drug: Polymer ratio: | 1:3 | |
| Coating weight: | 4 | mg/cm² |
| Coating thickness: | 30-50 | μm |
| Scaffold skeleton thickness: | 100-200 | μm |

| | |
|---|---|
| Therapeutic agent to be delivered: | ABT-348 or ABT-993 |
| Additional therapeutic agent to be delivered: | Paclitaxel or Zotarolimus |

A stent scaffold disclosed by U.S. Patent Application Publication No. 2011/0190872 can be used in this example.

A bioabsorbable scaffold prepared according to the above specification is deployed to the artery proximal to the tumor tissue and is used to treat HCC and colorectal liver metastasis and tumor in general.

### 6A2. Local Delivery of Small Molecule Therapeutic Agents Via Particulates/Vesicles TACE

TACE (transarterial chemoembolization) is a procedure in which the blood supply to a tumor is blocked (embolized) and chemotherapy is administered directly into the tumor. The procedure involves gaining percutaneous access to the hepatic artery using a catheter, identifying the branches of the hepatic artery supplying the tumor(s), selecting a blood vessel supplying tumor, injecting alternating aliquots of the chemotherapy dose and embolic particles, or particles containing the chemotherapy agent through the catheter. The total chemotherapeutic dose may be given in one vessel's distribution, or it may be divided among several vessels supplying the tumor(s).

Bioabsorbable microparticles or nanoparticles containing small molecule therapeutic agent can be delivered using TACE technique. In this example, paclitaxel loaded microparticles or nanoparticles of PLGA (50/50) are provided. The particles are injected into a blood vessel that supplies the tumor to be treated through a catheter. The particles block the blood supply to the tumor and release paclitaxel to the tumor.

Halofuganone and everolimus loaded microparticles or nanoparticles of PLGA(50/50) are also provided and injected into a blood vessel that supplies the tumor to be treated through a catheter. The particles block the blood supply to the tumor and release halofuganone or everolimus to the tumor.

### 6A3. Local Delivery of Biologics Via Hydrogel:

A hydrogel containing biologics is prepared according to the following.

| Sample No. | Gel | Biologics |
|---|---|---|
| 1 | PEG-PEG in situ crosslinkable hydrogel | anti-TNF |
| 2 | PEG-PEG in situ crosslinkable hydrogel | ABT-806 |
| 3 | PEG-PEG in situ crosslinkable hydrogel | BCL-2 inhibitor |

The biologics-containing hydrogel can be delivered by injection directly into a tumor tissue or the feeding artery proximal to the tumor tissue. This method can be used to treat Glioblastoma.

6B. Local Delivery of Therapeutic Agent as in 6A Combined with Systemic Therapy.

The local delivery method provides additive or synergistic effect with systemic therapy.

6B1. Local Delivery of Small Molecule Therapeutic Agent Via Bioabsorbable Scaffold as in 6A1 Combined with Systemic Therapy

A small molecule drug ABT-348 or ABT-993 is delivered via a drug eluting bioabsorbable scaffold as in 6A1. Additional small molecule drug paclitaxel or zotarolimus can be added to the drug eluting bioabsorbable scaffold.

As part of the treatment, a small molecule drug ABT-869 is delivered by a systemic means such as oral administration and intravenous injection or infusion. This method can be used to treat HCC and colorectal liver metastasis.

6B2. Local Delivery of Small Molecule Therapeutic Agents Via Particulates/Vesicles TACE as in 6A2 Combined with Systemic Therapy

Paclitaxel loaded microparticles or nanoparticles of PLGA (50/50) or halofuganone and everolimus loaded microparticles or nanoparticles of PLGA(50/50) are delivered using TACE technique as in 6A2. As part of the treatment, a small molecule drug ABT-869 is delivered by a systemic means such as oral administration and intravenous injection or infusion. The method can be used to treat HCC and colorectal liver metastasis.

6C. Systemic Drug Delivery from Local Implant.

This method allows the therapeutic agent to elicit a systemic response but be implanted in a vascular location (e.g., saphenous vein). This method also allows better bioavailability, lower systemic toxicity, and use of drugs that are hard to formulate for other systemic delivery means.

6C1. A small molecule drug ABT-348 is loaded on a drug eluting bioabsorbable scaffold as in 6A1. The scaffold is implanted in saphenous vein etc. Additional small molecule drug paclitaxel or zotarolimus can be added to the drug eluting bioabsorbable scaffold as in 6A1.

### Example 7

Anti-VEGF monoclonal antibody avastin, anti-EGRF antibody ABT-806, or small molecule drug ABT-869 is incorporated into bioabsorbable microparticles or nanoparticles as described in Example 3. The drug containing particles are delivered directly to a tumor tissue using a catheter.

Anti-VEGF monoclonal antibody avastin, anti-EGRF antibody ABT-806, or a small molecule drug ABT-869 is loaded on a drug eluting scaffold described in Example 6. The scaffold is implanted in an artery proximal to a tumor tissue.

Anti-VEGF monoclonal antibody avastin, anti-EGRF antibody ABT806, or a small molecule drug ABT-869 is mixed with a PEG/PEG in-situ crosslinkable hydrogel as described in Example 6. The hydrogel is injected to a tumor tissue.

### Example 8

The same type of rapid gelation hydrogel in Example 1 is used to embolize or clog the arteries feeding the tumor. The hydrogel injection immediately follows injection of embolization beads in the amount lower than that if used alone. The initial injection of embolic beads serves to plug the distal arterial bed. The hydrogel contains an anti-VEGF agent as well as embolization beads mixed within. The mixture of hydrogel and beads provides a more effective and complete seal of the arterial bed than the hydrogel alone or the beads alone thereby preventing unoccluded microcirculation that continues to supply the tumor and provides for dual drug delivery of cytotoxic and anti-VEGF compounds.

### Example 9

The same type of rapid gelation hydrogel in Example 1 is delivered directly to the tumor as a stand-alone local anti-VEGF therapy. For example, a needle balloon catheter can inject hydrogel with an anti-VEGF agent around the tumor (peritumoral) or within the tumor (intralesional) under X-ray guidance. The hydrogel will not only encapsulate and isolate the tumor but also provide a local sustained release of drug.

In the above examples, zotarolimus can be combined with an anti-VEGF therapy. In addition to zotarolimus, there are additional mTOR inhibitors that should be considered including sirolimus, biolimus, everolimus, deforolimus, and SAR-943. Of particular note is SAR-943 (Novartis) which is 10 to 100 fold more potent than zotarolimus. Given the greater potency of SAR-943, one could use less drug to obtain the same amount of inhibition or use the same or more drug to extend the duration of release.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method of treating a liver cancer in a subject in need thereof comprising:
deploying a bioresorbable polymer scaffold in the lumen of a blood vessel that directly services a diseased liver or a cancer tissue therein,
wherein the bioresorbable polymer scaffold comprises a polymer substrate and optionally a coating upon the substrate, wherein a first therapeutic agent is embedded or impregnated in the substrate, the coating if present, or both,
wherein a therapeutically effective amount of the first therapeutic agent is released from the scaffold upon the deployment thereof over a period of time;
wherein the first therapeutic agent is an anti-angiogenesis agent.

2. The method of claim 1, wherein the blood vessel is a feeding artery proximal to the diseased liver or the cancer tissue therein.

3. The method of claim 1, wherein the liver cancer is hepatocellular carcinoma (HCC), colorectal liver metastasis, or heptoblastoma.

4. The method of claim 1, further comprising a step of delivering embolic beads to the tumor, wherein the embolic beads are embedded with a radioactive isotope, a radioactive anti-tumor drug or a chemotherapy drug.

5. The method of claim 1, wherein the cancer tissue is in a state of hypoxia due to blockage of arterial blood supply by embolic beads embedded in the liver.

6. The method of claim 4, wherein the embolic beads are bioabsorbable.

7. (canceled)

8. The method of claim 1, wherein the anti-angiogenesis agent is selected from the group consisting of an anti-VEGF monoclonal antibody, an anti-EGFR monoclonal antibody, a small molecule anti-angiogenesis agent, and any combination thereof.

9. The method of claim 8, wherein the anti-VEGF antibody is Avastin, the anti-EGRF anti-body is ABT-806, the small molecule drug is selected from the group consisting of sorafenib, linifanib (ABT-869), ABT-348, and any combination thereof.

10. The method of claim 1, wherein the anti-angiogenesis agent is sorafenib or linifanib (ABT-869).

11. The method of claim 1, wherein the substrate or the coating or both further comprise a second therapeutic agent selected from the group consisting of mTOR inhibitors, anti-proliferative agents, anti-inflammatory agents, and anti-neoplastic agent.

12. (canceled)

13. (canceled)

14. The method of claim 11, wherein the second therapeutic agent is selected from the group consisting of paclitaxel, zotarolimus, everolimus, sirolimus, tacrolimus, biolimus, deforolimus, SAR-943, halofuganone, an anti-TNF agent, and any combination thereof.

15. The method of claim 1, wherein the substrate comprises a bioabsorbable polymer selected from the group consisting of poly(DL-lactide), poly(L-lactide), poly(D-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof, and a combination thereof.

16. The method of claim 1, wherein the coating is a polymer matrix comprising a bioabsorbable polymer selected from the group consisting of poly(DL-lactide), poly(L-lactide), poly(D-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof, and a combination thereof.

17. A drug eluting device comprising:
a bioabsorbale polymer scaffold which comprises a polymer substrate and optionally a coating upon the substrate,
a first therapeutic agent which is an anti-angiogenesis agent selected from the group consisting of an anti-VEGF antibody, an anti-EGFR antibody, a small molecule anti-angiogenesis drug, and any combination thereof;
optionally a second therapeutic agent selected from the group consisting of anti-proliferative agents, anti-inflammatory agents, and anti-neoplastic agent;
wherein the therapeutic agents are incorporated either in the polymeric substrate or the coating if present or both.

18. The drug eluting device of claim 21, which is a stent.

19. The drug eluting device of claim 17, wherein the anti-VEGF antibody is Avastin; the anti-EGRF anti-body is ABT-806; and the small molecule drug is selected from the group consisting of sorafenib, linifanib (ABT-869), ABT-348, and any combination thereof.

20. The drug eluting device of claim 17, wherein the first therapeutic agent is linifanib (ABT-869).

21. The drug eluting device of claim 17, comprising a second therapeutic agent selected from the group consisting of paclitaxel, zotarolimus, everolimus, sirolimus, tacrolimus, biolimus, deforolimus, SAR-943, halofuganone, an anti-TNF agent, and any combination thereof.

22. The drug eluting device of claim 17, wherein the substrate comprises a bioresorbable polymer selected from the group consisting of poly(DL-lactide), poly(L-lactide), poly(L-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof, and a combination thereof.

23. The drug eluting device of claim 17, wherein the coating is a polymer matrix comprising a bioresorbable polymer selected from the group consisting of poly(DL-lactide), poly(L-lactide), poly(L-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof, and a combination thereof.

24-45. (canceled)
